(19) United States
(12) Patent Application Publication
(10) Pub. No.: US 2011/0098624 A1
(43) Pub. Date: Apr. 28, 2011

(54) Method and apparatus for treating renal disease with hemodialysis utilizing pulsatile pump

(76) Inventors: Craig McCotter, Crozet, VA (US); Paul Bolin, Greenville, NC (US)

(21) Appl. No.: 10/467,755

(22) PCT Filed: Feb. 6, 2002
(86) PCT No.: PCT/US02/03602

§ 371 Date: Jun. 29, 2007

Related U.S. Application Data
(60) Provisional application No. 60/268,566, filed on Feb. 14, 2001.

(51) Int. Cl. A61M 1/16

(52) U.S. Cl. 604/5.04; 604/6.09

(57) ABSTRACT

A method of removing toxins from blood from a patient in need of such toxin removal includes: providing a countercurrent dialysis filter (20) having a blood compartment and a dialysate compartment separated from the blood compartment by a semi-permeable membrane; conveying blood from the patient through the blood compartment of a countercurrent filter (20) an back to the patient; and drawing dialysate from a reservoir (32) through the dialysate compartment of the countercurrent filter (20). At least one of the blood or dialysate experiences pulsatile flow. The steps are carried out such that blood toxins are drawn from the blood compartment through the semi-permeable membrane into the dialysate compartment.

### FIELD OF THE INVENTION

The present invention is directed generally to treatment of renal disease, and more specifically to treatment of end stage renal disease (ESRD) with hemodialysis.

### BACKGROUND OF THE INVENTION

Hemodialysis is a well-known treatment technique for ESRD, a condition in which the patient's renal system has essentially ceased to remove waste products and contaminants from the blood. Hemodialysis is a process that involves removing blood from the vasculature of a patient (usually a shunt or vein), purifying it with dialysate (a fluid that helps to remove toxins and return electrolytes to the blood), and returning the blood to the patient (usually through another vein).

Hemodialysis machines typically operate with separate compartments for blood and dialysate. In conventional systems, the compartments are separated by a semi-permeable membrane that allows selective diffusion; toxins are removed from the blood, and electrolytes are added to bring the electrolyte concentration of the blood to desired levels.

In many systems, the blood and dialysate compartments are arranged in a countercurrent flow exchange layout, with the blood traveling in one direction and the dialysate traveling in the opposite direction. One arrangement employs a large tube within which reside many smaller diameter tubules. Ordinarily, the large tube carries dialysate, and the smaller tubules carry blood. Blood is typically pumped through the tubules with a positive pressure pump (exemplary is a roller head pump), and the dialysate is typically drawn through the large tube with a roller head pump.

There are some shortcomings with such an arrangement. The relatively constant flow of the blood and dialysate can create “dead” regions (where flow essentially stops) and regions of laminar flow within the tube. In each of these regions the component exchange between the blood and the dialysate can be adversely affected, as fluid in the dead regions and the regions of laminar flow tends to have less surface area contact with the membrane, thereby reducing the efficiency of exchange. Also, the constant flow of dialysate can create “shunting” of dialysate in certain regions, which can lead to blood/dialysate mismatch. As such, it would be desirable to provide a hemodialysis system with improved efficiency of exchange.

### SUMMARY OF THE INVENTION

The present invention can address some of the shortcomings of prior systems by improving the efficiency of dialysis through the use of pulsatile flow. As a first aspect, the invention includes a method of removing toxins from blood from a patient in need of such toxin removal, comprising: providing a dialysis filter having a blood compartment and a dialysate compartment separated from the blood compartment by a semi-permeable membrane; conveying blood from the patient through the blood compartment of a filter and back to the patient; and drawing dialysate from a reservoir through the dialysate compartment of the countercurrent filter. At least one of the blood or dialysate experiences pulsatile flow. The steps are carried out such that blood toxins are drawn from the blood compartment through the semi-permeable membrane into the dialysate compartment.

As a second aspect, the invention includes an apparatus for performing hemodialysis on a subject in need of such treatment. The apparatus comprises: a dialysis filter having a blood compartment and a dialysate compartment separated from the blood compartment by a semi-permeable membrane; a first pump fluidly connected with the blood compartment that conveys blood from the patient through a blood compartment of a filter and back to the subject; and a second pump fluidly connected to the dialysate compartment. At least one of the first and second pumps is configured to induce pulsatile flow.

It has been discovered that the use of pulsatile flow in the blood or dialysate circuits during hemodialysis can provide a number of advantages. These include increased efficiency of transport, reduction in dead regions, and intermittent increased transmembrane pressure.

Objects of the present invention will be appreciated by those of ordinary skill in the art from a reading of the figures and the detailed description of the preferred embodiments which follow, such description being merely illustrative of the present invention.

### BRIEF DESCRIPTION OF THE FIGURES

The accompanying drawings, which are incorporated in and constitute a part of the specification, illustrate embodiments of the invention and, together with the description, serve to explain principles of the invention.

FIG. 1 is a schematic diagram of a hemodialysis apparatus of the present invention.

FIG. 2 is an enlarged schematic view of a hollow fiber artificial kidney (HFAK) included in the hemodialysis apparatus of FIG. 1.

FIG. 2A is a greatly enlarged schematic view of two blood tubules of the HFAK of FIG. 2.

FIG. 3 is a graph plotting urea concentration as a function of time in a two pool model of a dialysate.

FIG. 4 is a graph plotting creatinine concentration as a function of time collected in a dialysis study on dogs.

FIG. 5 is a graph plotting BUN concentration as a function of time collected in a dialysis study on a dog.

### DETAILED DESCRIPTION OF THE INVENTION

The present invention now will be described more fully hereinafter with reference to the accompanying drawings, in which preferred embodiments of the invention are shown and described. This invention may, however, be embodied in many different forms and should not be construed as limited to the embodiments set forth herein; rather, these embodiments are provided so that this disclosure will be thorough and complete, and will fully convey the scope of the invention to those skilled in the art. Like numbers refer to like components throughout.

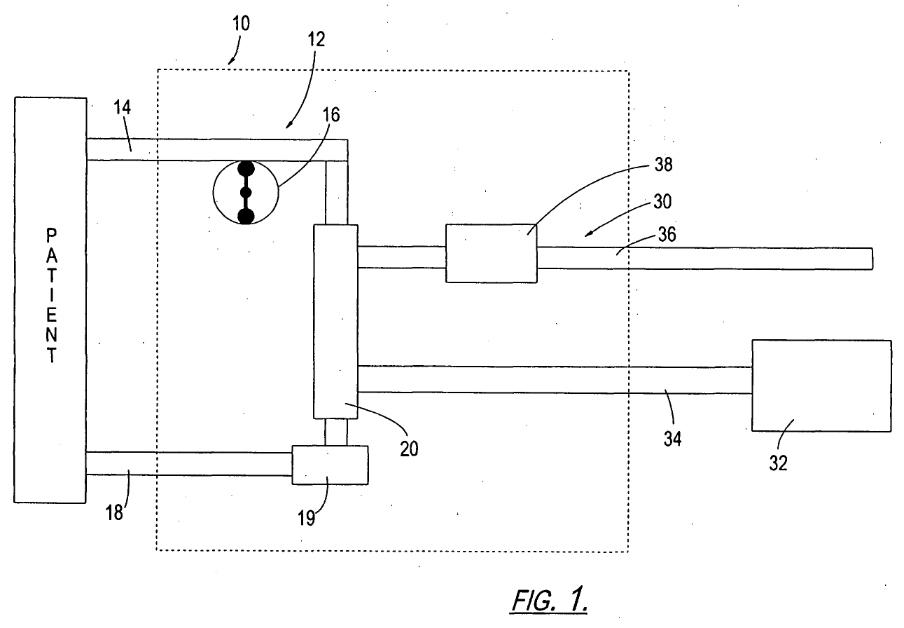

Referring now to FIG. 1, a hemodialysis apparatus, designated broadly at 10, is illustrated schematically. The hemodialysis apparatus 10 comprises a blood subsystem 12 and a dialysate subsystem 30, each of which will be described in greater detail below.

The blood subsystem 12, which as the name implies conveys blood through the hemodialysis apparatus 10, includes a blood entry conduit 14 that leads from a patient's artery (usually in the wrist), a roller pump 16 fluidly connected with the blood entry conduit 14, an HFAK 20 of conventional construction, and a blood exit conduit 18 leading from the HFAK 20 back to the patient's vein. A bubble trap 19 is located on the blood exit conduit 18 between the HFAK 20 and the vein of the patient to prevent bubbles created during the processing of the blood from entering the patient.

During operation, blood exits the patient through the blood entry conduit 14. The roller pump 16 forces the blood to the HFAK 20, wherein dialysis occurs as described below. The dialyzed blood then exits the HFAK 20 in the blood exit conduit 18, passes through the bubble trap 19 and returns to the patient's body.

The dialysate subsystem 30, which conveys dialysate into and from the HFAK 20, includes a dialysate reservoir 32 that contains dialysate, a dialysate entry conduit 34 leading from the dialysate reservoir 32 to the HVAK 20, a dialysate exit conduit 36 leading from the HFAK 20, and an in-line pulsatile pump 38 located on the dialysate exit conduit 36.

In operation, dialysate is pumped from the dialysate reservoir 32 through the dialysate entry conduit 34 and into the HFAK 20, wherein countercurrent exchange of components of the blood and dialysate. The spent dialysate is then pumped via the pulsatile pump 38 back to the dialysate reservoir 32.

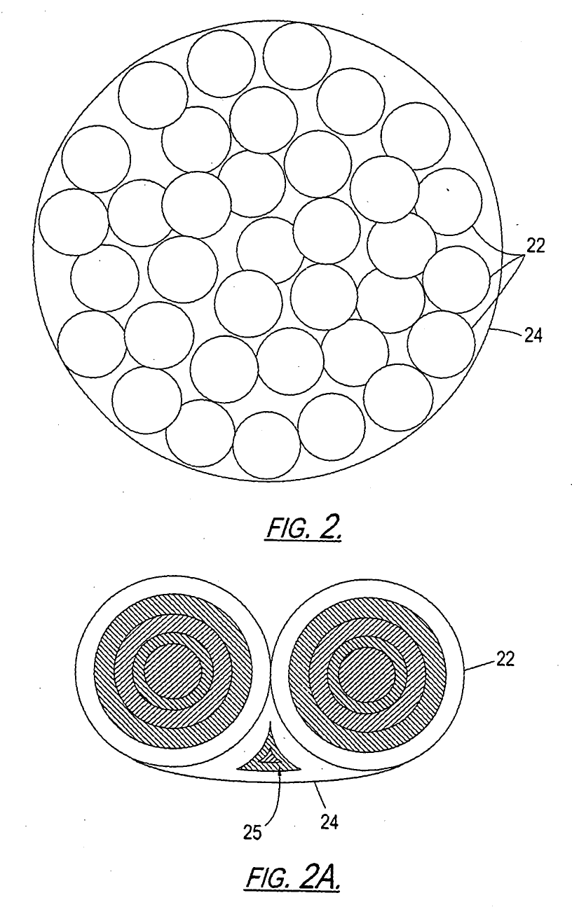

An exemplary HVAK 20 is illustrated in FIG. 2. The HVAK 20 includes a plurality of narrow blood tubules 22 that are enclosed within a dialysate casing 24. As the name implies, blood is conveyed through the blood entry conduit 14 into the blood tubules 22, and dialysate is conveyed from the dialysate entry conduit 34 into the dialysate casing 24. The blood tubules 22 are formed of a semi-permeable membrane material, such as polysulfone, that enables countercurrent exchange of components to occur between the blood and dialysate. The dialysate casing 24 is typically formed of an impervious material such as a plastic material.

The dialysate utilized in the dialysate subsystem 30 can be any dialysate known to those skilled in this art as being suitable for use in hemodialysis. An exemplary dialysate is available under the trade name Neutralyte from Fresenius Medical Care, Lexington, Mass.

The use of the pulsatile pump 38 (which can be a piston pump, a modified roller head pump, or the like) in the dialysate subsystem 30 can have the effect of producing pulsatile flow of dialysate within the dialysate subsystem 30. As used herein, “pulsatile flow” means flow that has a pulse pressure of 10 mm Hg or greater, and preferably when applied to blood means a pulse pressure of 30 mm Hg or greater. Preferably, the pulsatile flow is induced in the dialysate at a pulse rate of between about 30 and 100 cycles per minute, and more preferably at a rate of between about 50 and 80 cycles per minute. Those skilled in this art will recognize that the pulsatile pump may be used alone or in combination with a more constant flow pump.

Such pulsatile flow within the dialysate subsystem 30 can generate significant turbulence within the dialysate casing 24, which can reduce or eliminate the number of “dead” regions, where flow stops and exchange is minimal. A typical flow pattern in blood tubules 22 and the blood casing 24 is shown in FIG. 2A (darker areas represent regions of higher flow), with a dead region 25 being illustrated between two tubules 22. Turbulence can increase the exchange efficiency between the blood and dialysate by increasing the amount of surface area contact between the dialysate and the blood tubules 22. Also, the pulsation can produce bursts of increased transmembrane pressure, which additionally helps exchange. The increased energy introduced into the system can also enhance transport. Further, reduction of “dead” regions reduces the tendency of clotting, with secondary improvement in transport. As a result of these combined effects, the overall efficiency of hemodialysis (for example, increased clearance of urea and creatinine) can be significantly increased over systems that lack a pulsatile dialysate flow.

It should also be noted that the pump 16 of the blood subsystem 12 may also be a pulsatile pump, with many of the advantages described above for the dialysate subsystem 30 also being achievable with pulsatile flow in the blood subsystem 12. If a hemodialysis apparatus includes pulsatile pumps in both the blood and dialysate subsystems, the pulsatile pumps may be synchronous, such that they pump at the same rate and with matching amplitudes, or they may be dissynchronous, such that they pump at different rates and/or with mismatched amplitudes. Given that reduction of dead regions in the HFAK 20 can improve transport, it may be preferred to employ dissynchronous pulsatile pumps, as doing so may increase turbulence.

The invention and the advantages achievable therewith will now be described in greater detail in the following non-limiting examples.

### Example 1

### In Vitro Analysis of Pulsatile Flow of Dialysate

In vitro dialysis experiments were conducted using a pair of pools filled with liquid and performing dialysis on these liquids. Experimental blood and dialysate circuits were created that each included a two-liter reservoir connected to a roller head pump (Sarns 7400 MDX, available from Sarns, Inc., Ann Arbor, Mich.). The roller head pump was connected to a dialysis filter, which was in turn connected to the two liter reservoir to form a closed loop. All connections were made with ⅜ inch plastic tubing.

For the control apparatus, the dialysis machine used was a Fresenius 2008H dialysis machine (available from Fresenius USA, Lexington, Mass.), which employs a relatively constant flow pump. The experimental system employed a Fresenius F7NR dialysis filter (available from Fresenius USA, Lexington, Mass.) and a Sarns roller head pump that operated at 50 cycles per minute and at a pulse pressure of 80 mm Hg.

For each system, the two liter reservoir was filled with a mixture of tap water and urea (2 liters water to 9.8-10 g of urea) to be used as experimental blood. This mixture had a measured level of 95-100 mg/dL urea. Tap water was used as the experimental dialysate. Experimental blood and dialysate were then allowed to flow through the system at predetermined rates. Samples of fluid were drawn from the reservoir at 5 minute intervals for 35 minutes, and the urea level was measured with an Olympus AU640 instrument (available from Olympus Corp., Dallas, Tex.).

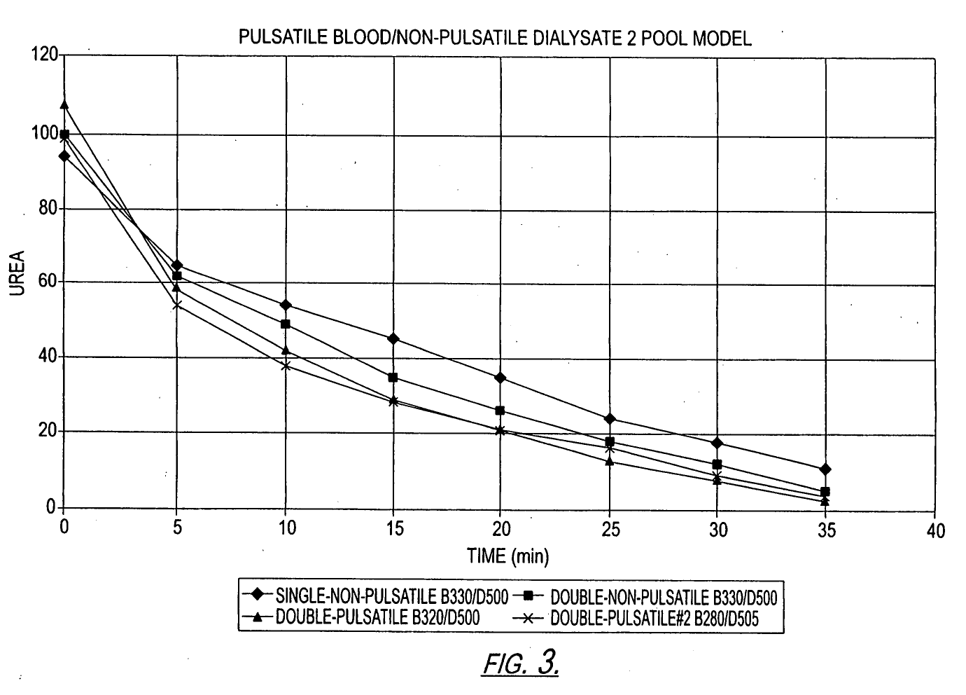

The results of the procedures can be seen in Tables 1 and 2 below and FIG. 3.

TABLE 1
Dialysate Flow -500 cc/min
Pulsatile Experimental Blood Compartment Only

| Blood Flow | | Urea | | | | |
|---|---|---|---|---|---|---|
| (cc/min) | Procedure | Baseline | 5 min | 10 min | 15 min | 20 min |
| 200 | Control Dialysis | 94 | 77 | 53 | 40 | 28 |
| | | | (18.1%) | (43.6%) | (57.4%) | (70.2%) |
| | Variable Speed | 97 | 65 | 39 | 23 | 14 |
| | Dialysis | | (33%) | (59.8%) | (76.3%) | (85.6%) |
| | % Greater Efficiency | | 45 | 27 | 22 | 18 |
| 250 | Control Dialysis | 97 | 64 | 42 | 25 | 15 |
| | | | (34.0%) | (56.7%) | (74.2%) | (84.5%) |
| | Variable Speed | 95 | 57 | 32 | 18 | 9 |
| | Dialysis | | (40.0%) | (66.3%) | (81.1%) | (6.6%) |
| | % Greater Efficiency | | 15 | 9.6 | 8.5 | 6.6 |
| 330 | Control Dialysis | 97 | 61 | 33 | 18 | 10 |
| | | | (37.0%) | (65.9%) | (81.4%) | (89.7%) |
| | Variable Speed | 94 | 56 | 27 | 14 | 7 |
| | Dialysis | | (40.0%) | (71.3%) | (85.1%) | (92.6%) |
| | % Greater Efficiency | | 7.5 | 7.6 | 3.7 | 3.0 |
| 400 | Control Dialysis | 113 | 75 | 53 | 33 | 20 |
| | | | (33.6%) | (53.1%) | (70.8%) | (82.3%) |
| | Variable Speed | 114 | 55 | 30 | 14 | 7 |
| | Dialysis | | (51.8%) | (73.7%) | (87.7%) | (93.9%) |
| | % Greater Efficiency | | 35.1 | 20.6 | 19.3 | 12.4 |

TABLE 2
Dialysate Flow-190 cc/min
Pulsatile Experimental Dialysate Compartment Only

| Blood Flow | | Urea | | | |
|---|---|---|---|---|---|
| (cc/min) | Procedure | Baseline | 5 min | 10 min | 15 min |
| 215 | | | | | |
| | Control Dialysis | 97 | 80 | 62 | 38 |
| | | | (17.5%) | (36.0%) | (60.8%) |
| | Variable Speed | 96 | 73 | 54 | 30 |
| | Dialysis | | (23.9%) | (43.7%) | (68.8%) |
| | % Greater | | 26.7 | 17.6 | 11.6 |
| | Efficiency | | | | |

As can be seen from Tables 1 and 2 and FIG. 3, the efficiency of hemodialysis increases significantly (as much as 35 percent) with the employment of a variable speed pump for pumping blood or dialysate.

### Example 2

The experimental apparatus used in Example 1 was employed again to perform dialysis on a dog with the following changes. A HB 500 filter was employed with the Century System III dialysis machine (both available from Gambro Corp., Lakewood, Colo.). Also, rather than tap water, a standard 3K dialysate was used to hemodialyze the dog. Pulsatile flow was induced in the blood subsystem with a roller head pump operating at 50 cycles/minute. In addition, both blood urea nitrogen (BUN) and creatinine levels were measured (BUN was measured with a Olympus AU640 instrument and creatinine was measured by a Jaffe assay).

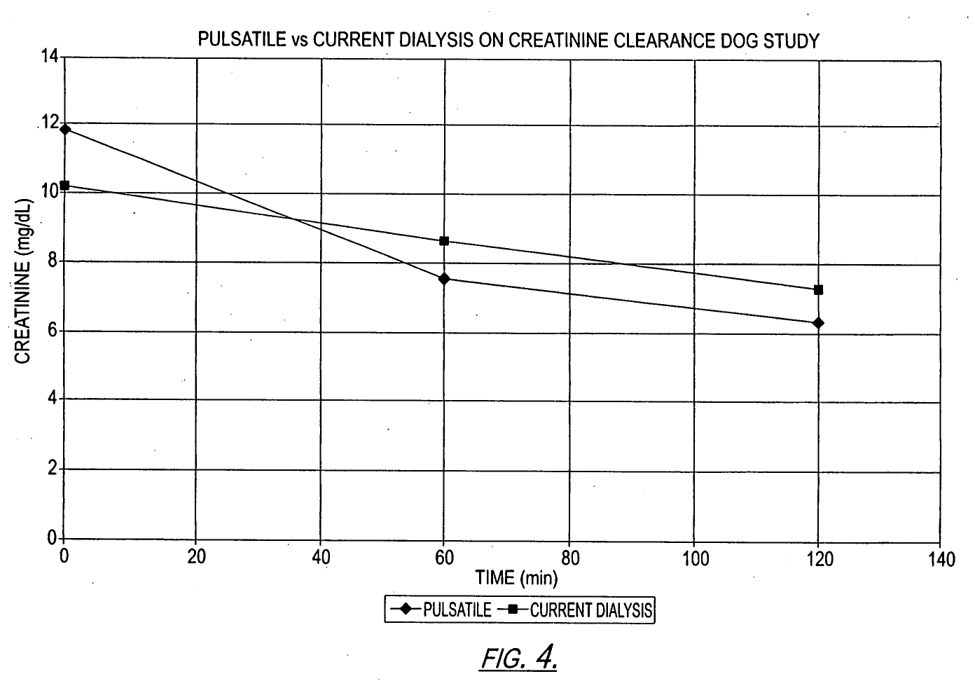

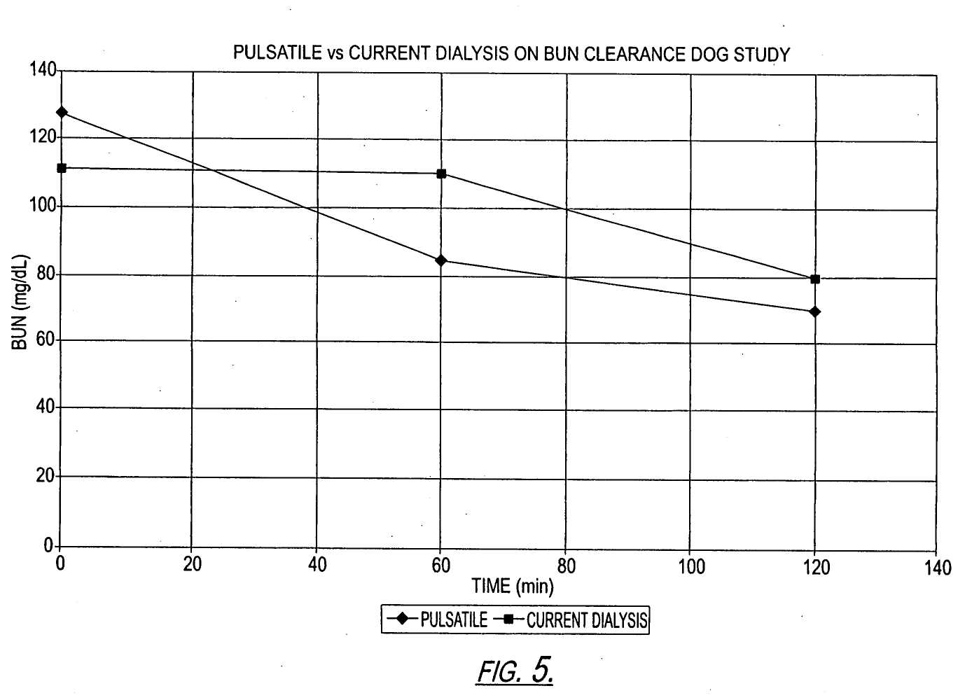

The results of the procedure are shown in Tables 3 and 4 and FIGS. 4 and 5.

TABLE 3
BUN Level

| Procedure | Baseline | 60 min | 120 min | Post 30 min |
|---|---|---|---|---|
| Control Dialysis | 110 | 110 (0.0%) | 80 (27.3%) | 97 |
| Variable Speed Dialysis | 126 | 84 (33.3%) | 69 (45.3%) | 73 |
| % Greater Efficiency | | | 39.8% | |

TABLE 4
Creatinine Level

| Procedure | Baseline | 60 min | 120 min | Post 30 min |
|---|---|---|---|---|
| Control Dialysis | 10.2 | 8.7 (14.7%) | 7.3 (28.5%) | 9.1 |
| Variable Speed | 11.8 | 7.7 (34.7%) | 6.3 (44.7%) | 7.4 |
| Dialysis | | | | |
| % Greater | | 56.7% | 36.3% | |
| Efficiency | | | | |

These results again indicate that the variable speed dialysis procedure is significantly more efficient than the control apparatus.

The foregoing is illustrative of the present invention, and is not to be construed as limiting thereof. The invention is defined by the following claims, with equivalents of the claims to be included therein.

What is claimed is:

1. A method of removing toxins from blood from a patient in need of such toxin removal, comprising:
providing a countercurrent dialysis filter having a blood compartment and a dialysate compartment separated from said blood compartment by a semi-permeable membrane;
conveying blood from the patient through the blood compartment of a filter an back to the patient; and
drawing dialysate from a reservoir through the dialysate compartment of the filter such that blood toxins are drawn from said blood compartment through the semi-permeable membrane into the dialysate compartment;
wherein at least one of the conveying and drawing steps is carried out with pulsatile flow.

2. The method defined in claim 1, wherein said drawing step comprises drawing the dialysate through the dialysate compartment with a pulsatile pump.

3. The method defined in claim 2, wherein said drawing step comprises drawing the dialysate through the dialysate compartment at a pulsatile pressure of greater than about 10 mm Hg.

4. The method defined in claim 1, wherein said drawing step comprises drawing the dialysate through the dialysate compartment at between 30 and 100 pulses per minute.

5. The method defined in claim 1, wherein said drawing step further comprises drawing the dialysate through the dialysate compartment with a roller head pump.

6. The method defined in claim 1, wherein said conveying step comprises conveying blood from the patient through the blood compartment with a constant flow pump.

7. The method defined in claim 1, wherein said providing step comprises providing a dialysis filter with a dialysate compartment comprising a dialysate casing and a blood compartment comprising a plurality of tubules that reside within the dialysate casing.

8. The method defined in claim 1, wherein said conveying step comprises conveying blood with a pulsatile pump.

9. The method defined in claim 1, wherein said conveying step comprises conveying the blood with a pulse pressure of greater than about 30 mm Hg.

10. An apparatus for performing hemodialysis on a subject in need of such treatment, comprising:
a dialysis filter having a blood compartment and a dialysate compartment separated from said blood compartment by a semi-permeable membrane;
a first pump fluidly connected with the blood compartment that conveys blood from the patient through a blood compartment of the filter and back to the subject; and
a second pump fluidly connected to the dialysate compartment;
wherein at least one of said first and second pumps is configured to induce pulsatile flow.

11. The apparatus defined in claim 10, wherein said second pump comprises a pulsatile pump.

12. The apparatus defined in claim 11, wherein said second pump is configured to induce a pulsatile pressure of greater than about 10 mm Hg in dialysate in the dialysate compartment.

13. The apparatus defined in claim 10, wherein said dialysate compartment comprises a dialysate casing and said blood compartment comprises a plurality of blood tubules residing within said dialysate casing.

14. The apparatus defined in claim 10, further comprising a third constant flow pump fluidly connected to said second pump.

15. The apparatus defined in claim 10, wherein said second pump is configured to provide pulsatile flow at a rate of between about 30 and 100 cycles per minute.

16. The apparatus defined in claim 10, wherein said first pump and said second pump are pulsatile pumps.

15. The apparatus defined in claim 10, wherein the pulsatile pressure induced by said first or second pump is at least 30 mm Hg.

16. The apparatus defined in claim 10, wherein the pulsatile pressure induced by said first pump is at least 30 mm Hg.
